(19) United States
(12) Patent Application Publication
(10) Pub. No.: US 2026/0090833 A1
(43) Pub. Date: Apr. 2, 2026

(54) TISSUE SEALING INSTRUMENT

(71) Applicant: Erbe Elektromedizin GmbH, Tuebingen (DE)

(72) Inventors: Volker MAYER, Tuebingen (DE); Rolf WEILER, Tuebingen (DE); Tanja HEIDER, Kirchentellinsfurt (DE); David LOESER, Rottenburg am Neckar (DE); Marcus AMANN, Balingen Engstlatt (DE); Markus PAESCH, Albstadt (DE); Yasar KELES, Burladingen (DE)

(21) Appl. No.: 19/334,194

(22) Filed: Sep. 19, 2025

(30) Foreign Application Priority Data
Sep. 27, 2024 (EP) 24203277.9

(51) Int. Cl. A61B 18/14; A61B 18/00

(52) U.S. Cl. CPC A61B 18/1445; A61B 2018/00083; A61B 2018/00601; A61B 2018/0063; A61B 2018/1452

(57) ABSTRACT

The tissue sealing instrument according to the invention comprises two jaws that are both provided with sealing electrodes. While one of the jaws comprises a cutting electrode, the other comprises an elastic counter support. The cutting electrode is held in a flexible plastic body that is seated in a narrow groove. On the contrary, counter support is seated in a comparably substantially wider recess. Accordingly, counter support is wider (preferably considerably wider) than insulating body comprising the cutting electrode. This definition of different dimensions allows a wide scope for defining desired characteristics of the tissue sealing instrument.

This application claims priority to European Patent Application No. 24203277.9, filed Sep. 27, 2024, the entirety of which is incorporated herein.

The invention refers to a tissue sealing instrument, particularly a forceps-like instrument having sealing electrodes at its two jaws and having an immobile cutting electrode.

Tissue sealing instruments of the indicated type are in general known and available on the market for the open surgical use, for the laparoscopic use or also for the endoscopic use. Examples for configuration of such instruments and particularly their head, substantially comprising the two jaws in addition to sealing and cutting electrodes, can be taken from EP 2 992 849 B1, for example. Such instruments comprise two jaws, whereby at least one of which is movably supported in order to hold tissue together with the other jaw in the type of forceps. While in one of the jaws an insulating body is arranged holding a cutting electrode, the other jaw comprises a counter support that itself is elastically soft and urges the tissue against the cutting electrode.

The instrument serves particularly for sealing and separating of vessels, for example blood vessels. In order to hold the two ends of a separated blood vessel also during sealing that has to be carried out yet reliably in the instrument, on both sides of cutting electrode tissue holding spaces are formed in which the bulge-like ends of the separated vessel are held in form-fit manner as long as the instrument is closed.

A similar instrument is also known from EP 3 138 522 B1. This instrument is characterized by a flexible counter support made of silicone that folds around the cutting electrode during a tissue cut.

Additional tissue sealing instruments having a cutting electrode are known from EP 1 632 192 A1, EP 2 409 653 B1, U.S. Pat. No. 8,679,115 B2 and EP 1 711 116 A2.

Particularly in instruments that have to be inserted through a limited channel, for example the working channel of an endoscope or through a trocar or the like, into the body of a patient the construction width of the instrument and thus also its sealing tool is limited. In addition, the jaws of the instrument shall be typically curved laterally in order to simplify the work on organs, that have naturally curved surfaces.

Under all these circumstances the tissue shall be securely held with the instrument being closed in order for the tissue not to be able to escape prior to termination of the treatment process.

Starting therefrom it is the object of the invention to provide an improved instrument.

This object is solved by means of a tissue sealing instrument according to claim 1:

The tissue sealing instrument according to the invention comprises two jaws that are movable toward and away from each other in the type of forceps. In one of the jaws a groove is provided in order to therein arrange an insulating body having a cutting electrode held in the insulating body. The other jaw also comprises a groove-like recess in which a flexible counter support for the cutting electrode is arranged. The particularity of the invention is the circumstance that the recess and together with it the counter support is wider, preferably substantially wider than the width of the groove. In doing so, the possibility is provided to arrange the cutting electrode in a flexible insulating material so that the cutting electrode can slightly flex laterally without becoming unstable thereby. The wider recess for the counter support allows to create tissue holding spaces that are spacious on one hand, however result on the other hand in that tissue held in the holding space is put under pressure and retained, particularly due to material displacement effects of the counter support.

Preferably the elastic counter support spans in its width not only the cutting electrode and the insulating body holding the latter, but additionally the distance between the insulating body and the lateral coagulation electrodes. The surfaces provided in this distance are preferably rigid, so that they unambiguously define the position of the tissue relative to the cutting electrode. This can support the quality of the cut as well as the reliability of the anchoring of the bulge-like tissue ends in the tissue holding spaces. Also due to this design the stability of the jaw holding the cutting electrode is supported.

The sealing electrodes are arranged on the two jaws, preferably on the edge. In this case they follow the outer contour of the jaw. In doing so, the lateral extension of the tissue holding spaces is maximized and the available space inside the instrument is used optimally.

The sealing electrodes of the jaw extend preferably respectively originating from a hinge side end section toward a distal end of the jaw. The sealing electrodes of each jaw can be connected at their distal ends among each other. Particularly, the sealing electrodes can be formed by one single seamlessly continuous, substantially U-shaped component. It is thereby advantageous if one of the sealing electrodes of each jaw extends from an area near the joint to a distal end area first along a straight line, to which a section with a small radius adjoins. On the contrary, the other of the sealing electrodes extends from the area near the joint to the distal end area in an arc having a comparably (much) larger radius. This arc is preferably arranged, so that the sealing electrodes have their minimum distance toward one another at a position, that is located between the area near the joint and the distal end area or is located adjoining the distal end area. In this manner particularly in the end area near the joint as well as in the distal end area a width of the space enclosed between the sealing electrodes is provided, so that biological tissue can be particularly reliably held therein. The reliable holding of the tissue in the area next to the joint and in the end area is of particular importance in order to avoid unintentional pulling of the tissue ends out of the closed tool. Also, this configuration supports the uniformity of the effect achieved on the tissue along the length of the cutting electrode and the sealing electrodes. This shape of the sealing electrodes is advantageous independent from the width of the groove and the width of the recess as well as their conformity or non-conformity. In addition, this shape of the sealing electrodes is advantageous in combination with the shape of the cutting electrode explained in relation to embodiments of the invention described in the following. This is again independent from the width of the groove, the width of the recess, the shape of the sealing electrodes as well as their conformity or non-conformity.

Preferably the recess for the counter support follows the shape of the sealing electrodes of the second jaw. On the contrary, the groove of the first jaw does not follow the shape of the sealing electrodes. Rather the groove is preferably configured with parallel flanks so that the groove flanks have varying distances to the adjacent sealing electrodes. This creates the possibility of simple manufacturing, particularly with regard to the creation of the groove, and concurrently provides the possibility of making the shape of the sealing electrodes independent from the groove. The sealing electrode can thereby have a single arc-shaped curvature or also a double curvature (slight S-shape). This feature can also be provided in a tool in which the recess has the same width as the groove, wherein the tool apart therefrom coincides with the described tool according to the invention. In this case the recess and the groove are aligned with one another, so that the counter support and the cutting electrode are exactly placed in line above each other during closing. However, it also applies here that the groove can have varying distances to the sealing electrodes along its length. For example, the groove or the cutting electrode can have a minimum distance to one sealing electrode and a maximum distance to the other sealing electrode at an approximately center position of its longitudinal extension. In addition, the distances of the groove toward the two sealing electrodes along their longitudinal extension can vary in different manner. For example, the distance to one of the sealing electrodes can increase locally while it decreases locally at the same position toward the other electrode.

Preferably the insulating body is configured in a flexible manner, whereby it comprises a first section held in the groove and a second section projecting out of the groove. The cutting electrode thereby extends through the second section into the first section, whereby also in case of a very slim configuration of the insulating body, a good lateral stability of the cutting electrode is obtained and the electrical insulation is guaranteed. Thereby the cutting electrode extends into the groove, so that the part of the cutting electrode having an accessible cutting edge is positioned above the groove and a part of the cutting electrode is positioned inside the groove. The part of the cutting electrode projecting out of the groove can be covered on its two flat sides by the insulating material of the insulating body and can thus be electrically insulated. Thereby the groove can extend from the area near the joint toward the distal area in an arc-shaped manner (arc that is curved only once) or also in an S-shaped manner (arc that is curved twice) for holding the insulating body. As required, however, the groove can be provided in one section, particularly its distal end, with groove walls that extend toward one another.

It is additionally advantageous if the counter support has a section with which it is supported on the bottom of the groove, preferably approximately centrally. In combination with optionally providable empty spaces between the jaw and the counter support the flexibility of the counter support and the force (particularly the distance-dependent progress of the force) with which the counter support presses against the tissue on both sides of the cutting electrode, can be adjusted appropriately. Particularly an initially high force can already be achieved in case of a slight deformation of the counter support. Thereby it is additionally advantageous with regard to the manufacturing and the fixation of the counter support inside the jaw if the counter support directly adjoins the sealing electrodes and thus insulates the latter against held biological tissue. In addition, due to the direct connection of the counter support to the inner flanks of the sealing electrode and the electrical insulation of the flanks resulting therefrom, creeping currents are effectively avoided. Due to the support of the counter support on the bottom of the recess, the connection between the counter support and the sealing electrode is maintained free from forces so that an adhesive or glue connection created there is not significantly affected. A distance between the counter support and the jaw can be configured directly where the counter support adjoins the sealing electrode. This distance creates a decoupling of the counter support from the connection to the sealing electrode with regard to forces and thus supports to the endurance of the substance bond connection at this position.

Further details of advantageous embodiments of the invention are subject of the description or the dependent claims as well as the complementing drawing. The drawing shows:

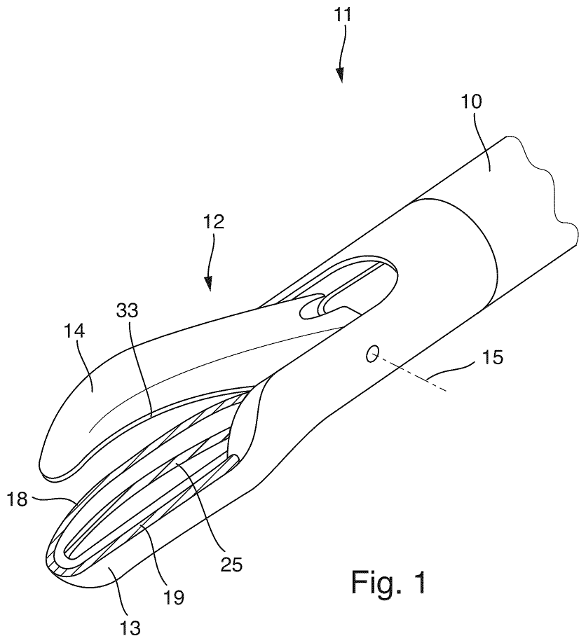

FIG. 1 shows a perspective view of the instrument according to the invention with its tool part in a partial perspective illustration,

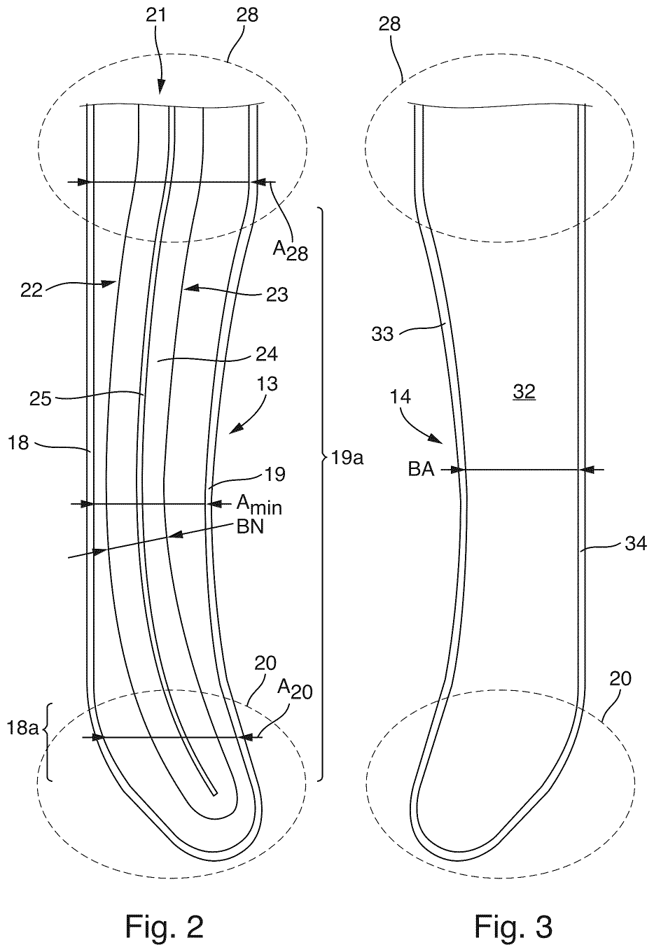

FIGS. 2 and 3 the two jaws of the instrument according to FIG. 1 are shown in a top view respectively,

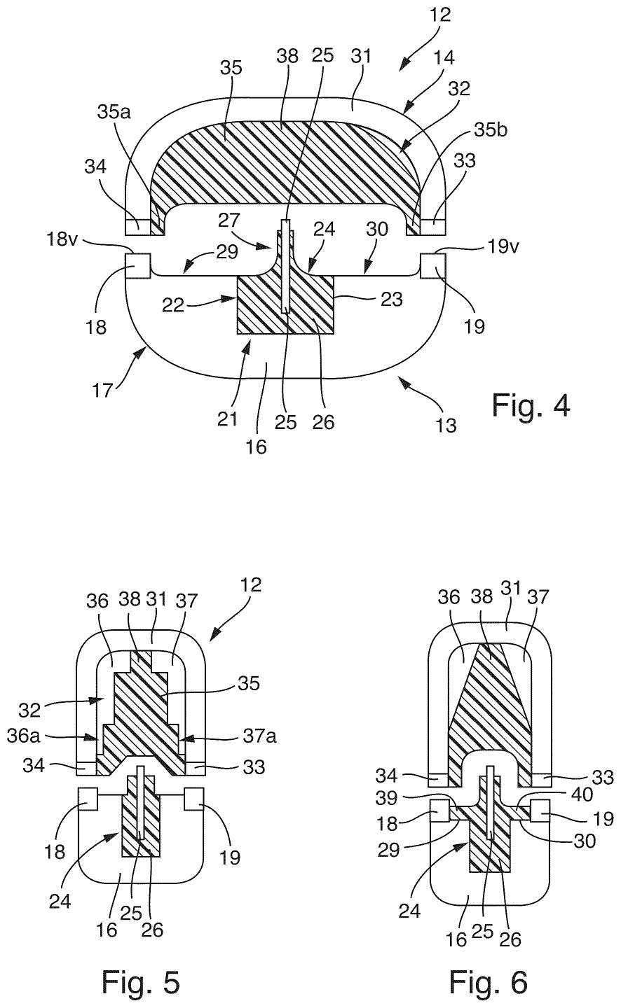

FIG. 4 the jaws of the instrument according to FIG. 1 are shown in a cross-section view at the position A₂₈ of FIG. 2,

FIGS. 5 and 6 show additional embodiments of the jaws of the instrument according to the invention in cross-section illustration at the position A₂₈,

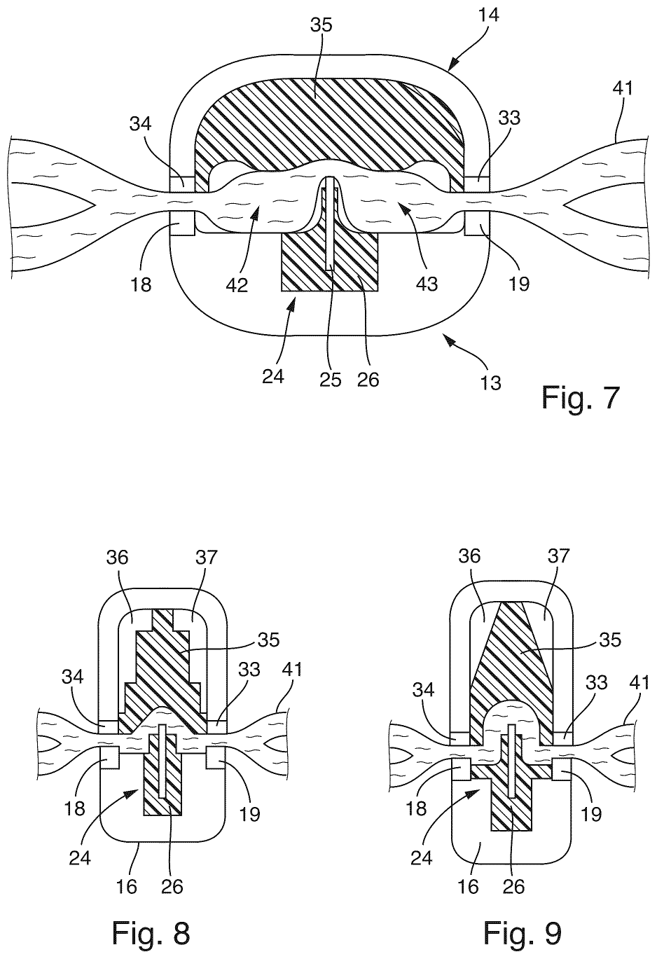

FIG. 7 shows the jaws according to FIG. 4 during sealing and separation of a vessel,

FIG. 8 shows the jaws according to FIG. 5 during sealing and separation of a biological vessel, and

FIG. 9 shows the jaws according to FIG. 6 during sealing and separation of a vessel.

In FIG. 1 a tool 12 is illustrated that is attached at a distal position on a shank 10 of a sealing instrument 11. A first jaw 13 and a second jaw 14 that are pivotably arranged toward and away from each other in the type of forceps, are part of this tool. For this purpose, at least one of the two jaws 13 and 14, in the present embodiment the second jaw 14, is pivotable relative to the first jaw 13 by means of a hinge device indicated by its pivot axis 15, wherein the first jaw 13 is immovably arranged. Alternatively, also both jaws 13 and 14 could be pivotably arranged toward and away from each other. The hinge device can be realized by one or two pivot bearings, a slotted guide, a spring hinge or the like.

The tissue sealing instrument 11 serves particularly for closing, sealing and cutting of vessels, for example blood vessels, but can also be used for other surgical actions, for example preparation of organs or other biological tissue.

For explanation of the further construction of the jaws 13 and 14 reference is made to FIGS. 2, 3 and 4. The first jaw 13 is formed by a stiff support part 16, for example made of metal, that can have an electrical insulation at its outer side 17. Alternatively support part 16 could also be partly or entirely made of a mechanically stable, only slightly flexible or inflexible and electrically insulating plastic. Also support part 16 can be a composite part and can be made from a metal inlay overmolded with plastic, for example.

Along its two edges the support part 16 of first jaw 13 can be provided with sealing electrodes 18 and 19 that can be connected via not further illustrated lines to an electrical generator. The two sealing electrodes 18 and 19 can be connected physically and electrically with each other in a distal end area 20 of first jaw 13, as illustrated in FIG. 2. Alternatively, however, also separate sealing electrodes 18 and 19 can be provided that can have equal or different potentials and that are not physically connected in the distal end area 20.

The first jaw 13 comprises a groove 21 between sealing electrodes 18 and 19, whereby the groove 21 is limited by groove flanks 22, 23. The groove flanks 22, 23 are preferably arranged with constant distance and thus parallel to each other as apparent from FIGS. 2 and 4, wherein its distance defines the groove width BN. Preferably, the groove width BN does not vary along the length of groove 21 or only in a section, preferably in a distal end area thereof. The groove 21 preferably comprises a rectangular or square cross-section. It is, however, also possible to provide groove 21 with a trapezoidal cross-section, so that it is narrower at the bottom than at its mouth facing the other jaw 14.

Inside groove 21 an insulating body 24 is arranged that is made from silicone, for example, or however also from another insulating material, particularly plastic. Preferably a plastic is used that comprises a noticeable flexibility or elasticity as well as a high creeping current strength. This improves the mechanical as well as the electrical function of the tissue sealing instrument 11 considerably.

In the insulating body 24 a cutting electrode 25 is arranged made from a thin sheet metal strip. The latter can have one or more openings through which the insulating body 24 passes in order to reliably anchor cutting electrode 25 inside insulating body 24. The cutting electrode 25 is covered on its two flanks by the insulating body 24 and thus electrically insulated. The insulating body thus forms a wall erecting in direction toward jaw 14, wherein at the top of the wall the cutting electrode 25 is uncovered. There, only a narrow face edge of cutting electrode 25 as well as optionally a small strip-shaped lateral area adjoining the face edge is uncovered. This lateral area is preferably not wider than the width of the face edge.

The insulating body 24 comprises a first section 26 located inside groove 21 and a second section 27 projecting from the first section 26 forming an erecting wall. The cutting electrode 25 extends through the second section 27 and into the first section 26.

Relating to the shape of the first jaw 13 in longitudinal direction reference is made to FIG. 2. From an area 28 near to joint to the distal end area 20 sealing electrode 18 extends along a largely straight line. Adjoining thereto inside the end section 20 or in proximity thereto an arc section 18a adjoins having a high curvature. On the contrary, the sealing electrode 19 extends originating from the area 28 near the hinge in an arc having low curvature. This curvature is of lower amount than the curvature in the area 18a. In the progress of area 19a the sealing electrode 19 extends toward the sealing electrode 18 and then away from the sealing electrode 18. For example, as illustrated in FIG. 2, the minimum distance Aₘᵢₙ between the sealing electrodes 18 and 19 can be located between the area 28 near the hinge and the distal end area 20 or also close to or inside the distal end area 20. Thus, the distance A₂₈ in the area 28 near the hinge and the distance A₂₀ in the distal end area 20 are preferably longer than the distance Aₘᵢₙ measured therebetween. The distances A₂₀, Aₘᵢₙ and A₂₈ have to be always measured transverse (orthogonal) to the linearly stretched sealing electrode. The measurement direction coincides with the direction in which tissue pulling forces are predominantly acting. In the distal end area 20 the sealing electrodes 18 and 19 approach each other in order to transition into one another at the distal end. The size of the area 28 near the hinge and the distal end area 20 can be defined in different ways. For example, the end area near the hinge can end where the linear section of sealing electrode 19 ends. The distal end area 20 can start where the linear section of sealing electrode 18 ends. Independent therefrom the area having the minimum distance Aₘᵢₙ is in each case between the areas 20, 28.

The groove 21 comprises a different extension. The groove 21 can follow an arc having minimum curvature in the progress of which it slightly approaches the straight sealing electrode 18 approximately centrally between the area 28 near the hinge and the distal end area 20 without dropping below the minimum insulation distance. Thereby it can follow an arc curved only once or can also be curved in a slightly S-shaped manner, which can result from arranging the cutting electrode 25 in the area 28 near the hinge at least in sections parallel to the cutting electrode 18. At least in most of the embodiments of the invention it is characteristic that cutting electrode 25 has a varying distance toward at least one of the sealing electrodes 18, 19, here toward the sealing electrode 18. Starting in the area near the hinge this distance is at first large. Further away from the area 28 near the hinge, approximately centrally of jaw 13, the distance is minimum and it is again slightly larger in the distal end area 20. This electrode configuration can also be used in tools in which the groove 21 and the recess 32 have coinciding widths and shapes. Apart therefrom, for such tools the remaining description applies accordingly.

As apparent from FIG. 4, on both sides of insulating body 24 surface areas 29, 30 of first jaw 13 are provided that adjoin insulating body 24 on one side and sealing electrodes 18 and 19 on the other side. These surface sections 29 and 30 can be metallic uncovered and can be in electrical contact with sealing electrodes 18, 19. It is however preferred if these surface sections 29, 30 are provided with an insulating coating or cover, for example a ceramic cover, a plastic cover (for example made from parylene) or the like. Also support part 16 can be made of plastic (with or without metal inlay), so that a separate insulation is unnecessary. The surface areas 29, 30 can be offset backwardly, that means located deeper with reference to FIG. 4, relative to sealing surfaces 18v, 19v provided on electrodes 18, 19. Further, the sealing areas can adjoin insulating body 24 in a stepless manner.

The sealing surfaces 18v, 19v of sealing electrodes 18, 19 illustrated in FIG. 4 are configured in a “narrow” manner. This means that their width that is to be measured in the drawing plane in FIG. 4 is smaller, preferably remarkably smaller, than the distance between the sealing electrode 18 and the cutting electrode 25 that is to be measured in the same direction. The same applies for the sealing electrode 19. This requirement applies at least for a section of sealing electrode 18, 19 or preferably for the entire length thereof. This configuration results in a narrow but reliable sealing seam on the tissue and provides in addition a large tissue holding space in which the tissue is secured during treatment.

The second jaw 14 comprises a support part 31 that in turn limits a recess 32 (FIG. 3) in the type of a groove. The support part 31 can again be made of metal or also plastic or a metal-plastic composite part. On its outer edge it supports the sealing electrodes 33, 34 that extend from the area 28 near the hinge to the distal end area 20. The sealing electrodes 33, 34 following the contour of support part 31 are mirror-symmetrically arranged relative to the sealing electrodes 18, 19. If the jaws are closed the sealing electrode 34 is congruent with sealing electrode 18. In addition, the sealing electrode 33 is congruent with sealing electrode 19. In so far, the description of the extension and the shape of the sealing electrodes 18, 19 applies accordingly in a mirror-symmetrical manner for the sealing electrodes 33, 34. In addition, the sealing electrodes 33, 34 can be connected with each other electrically and physically in the distal end area 20.

The recess 32 has a width BA, which can vary along the longitudinal extension of the second jaw 14. At each point it is, however, wider than the width BN of the groove 21. The width BA preferably coincides with the distance between sealing electrodes 33, 34. Thus, sealing electrodes 33, 34 and recess 32 have the same contour. However, it is indicated that the contour of sealing electrodes 33, 34 and recess 32 can also be defined different from one another. The distance between the sealing electrodes 33, 34 and its extension along the length of the jaw 14, therefore, coincides with the distance and the extension of sealing electrodes 18, 19—they are largely congruent.

Inside recess 32 a counter support 35 is arranged consisting of a flexible plastic. This preferably consists of a flexible plastic material, for example silicone. Preferably the counter support 35 is a closed monolithic body, which is free from hollow spaces. The counter support 35 is deformable, however cannot be compressed considerably. This means that in the preferred embodiment its volume cannot be reduced considerably due to the clamping forces acting in the tool.

The counter support 35 can completely fill recess 32 or, as indicated in FIG. 4, can leave smaller air pockets unfilled, so that counter support 35 can carry out a displacement movement corresponding to the size of the air pockets in case of deformation. In addition, counter support 35 extends with extensions 35a, 35b on the inner sides of sealing electrodes 33, 34 to which it is attached. The sealing electrodes 33, 34 are thereby electrically insulated toward the tissue holding spaces. Due to the support of section 38 of counter support 35 on the bottom of recess 32, the connection positions between the extensions 35a, 35b and the inner sides of sealing electrodes 33, 34 are largely free from forces, so that the existing connection is not or nearly not stressed or even affected.

Numerous modifications can be made to the embodiment of tool 12 described so far. For example, the tool 12 according to FIG. 5 can be configured considerably narrower. Air pockets provided as an option between support part 31 and counter support 35 can be used to specifically control deformation of the counter support during use. However, counter support 35 comprises a section 38 that is directly supported on the bottom of recess 32, as already in the embodiment according to FIG. 4. The air pockets 36, 37 can in addition extend with sections 36a, 37a up to the connection locations between counter support 35 and the sealing electrodes 33, 34, as depicted in FIG. 5 by way of example. In doing so, the connection locations can be relieved from mechanical shear and traction forces.

Also, in the embodiment according to FIG. 6, air pockets 36, 37 (not illustrated) and the supporting section 38 can be provided, which is why the description above applies accordingly based on similar reference signs. A particularity of the embodiment according to FIG. 6 is the insulating body 24, which comprises extensions 39, 40 projecting over surface section 29, 30. Apart therefrom, the remaining description of preceding embodiments applies accordingly.

In all embodiments sealing electrodes 18, 19 can be inserted in a respective cavity of support part 16. This simplifies the assembly and allows a precise positioning of sealing electrodes 18, 19.

It is in addition possible to provide the lateral walls 22, 23 of groove 21 with one or with multiple recesses into which the insulating body 24 extends with respective projections. These recesses can be arranged along one of the two side walls or also along both side walls 22, 23. This allows the specific definition of a lateral flexibility of electrode 25. The electrode 25 can also have lateral extensions at its edge located inside groove 21. These extensions can extend together with the insulating body 24 into lateral recesses if provided. This measure allows a particularly good lateral stabilization of cutting electrode with concurrent use of a highly flexible plastic for the insulating body 24.

The tissue sealing instrument 11 described so far operates as follows:

During use tissue is held between the jaws 13, 14 as illustrated in FIG. 7, for example. The sealing electrodes 33, 34 are thereby connected to one pole of a generator while the sealing electrodes 18, 19 are connected to another pole of the generator. The biological tissue held and compressed between the jaws 13, 14, for example the vessel 41, is therefore fusioned and coagulated between sealing electrodes 18, 33; 19, 34. Thereby cutting electrode 25 presses the biological tissue against counter support 35 that can dodge the pressure. However, in return it presses against the tissue in a dodge movement on both sides of the cutting electrode 25. The tissue is held in the tissue holding spaces 42, 43 formed on both sides of cutting electrode 25. Due to the dodge movement of counter support 35, that is due to the retreat from the tissue 41, space is made available for tissue 41 in the holding spaces 42, 43. In this manner the ends of the tissue 41, for example the vessel, are well retained in the instrument and are secured from escaping unintentionally. Concurrently to the tissue sealing between the electrode pairs 18/34, 33/19 or temporally offset, the cutting electrode 25 is applied with current so that this tissue 41 is severed.

In case of narrower instruments, as illustrated in FIGS. 8 and 9, the conditions are similar. However, there, due to the air pockets 36, 37, dodging of counter support 35 can also be made possible into the air pockets in order not to allow the pressure onto the tissue 41 to increase excessively.

The tissue sealing instrument 11 according to the invention comprises two jaws 13, 14 that are both provided with sealing electrodes 18, 19, 33, 34. While one of the jaws comprises a cutting electrode 25, the other comprises an elastic counter support 35. The cutting electrode 25 is held in a flexible plastic body 24 that is seated in a narrow groove 21. On the contrary, counter support 35 is seated in a comparably substantially wider recess. Accordingly, counter support 35 is wider (preferably considerably wider) than insulating body 24 comprising the cutting electrode 25. This definition of different dimensions allows a wide scope for defining desired characteristics of the tissue sealing instrument 11.

1. A tissue sealing instrument, the tissue sealing instrument comprising:
a first jaw and a second jaw that are pivotably supported by means of a bearing device toward and away from each other and that comprise sealing electrodes, respectively,
wherein the first jaw comprises a groove between the sealing electrodes that is limited by means of two opposite flanks, a distance from each other defines a first width (BN), wherein inside the groove a cutting electrode is held in an insulating body, and
wherein the second jaw comprises a recess between the electrodes, wherein the recess is limited by means of two opposite lateral surfaces and in which a flexible counter support for the cutting electrode is arranged, wherein a distance of the lateral surfaces from each other defines a second width (BA),
wherein the first width (BN) is smaller than the second width (BA).

2. The instrument according to claim 1, characterized in that the sealing electrodes of the first jaw are arranged on an edge thereof.

3. The instrument according to claim 1, characterized in that the sealing electrodes of the second jaw are arranged on an edge thereof.

4. The instrument according to claim 1, characterized in that the sealing electrodes of the first jaw are arranged on both sides of the groove extending toward a distal end area.

5. The instrument according to claim 4, characterized in that the sealing electrodes of the first jaw are connected with each other in the distal end area.

6. The instrument according to claim 1, characterized in that the sealing electrodes of the second jaw are arranged on both sides of the recess extending toward a distal end area.

7. The instrument according to claim 6, characterized in that the sealing electrodes of the second jaw are connected with each other at the distal end area.

8. The instrument according to claim 1, characterized in that one of the sealing electrodes of each jaw extends from an area near the bearing device to a distal end area along a straight line, while an other of the sealing electrodes extends in an arc from the area near the bearing device to the distal end area, wherein the arc is arranged so that a minimum distance (Aₘᵢₙ) of the sealing electrodes from each other is defined at a position between the area near the bearing device and the distal end area or at a position adjoining the distal end area.

9. The instrument according to claim 1, characterized in that the groove comprises an arc-shaped curved extension.

10. The instrument according to claim 1, characterized in that the first width (BN) is constant along a length of the groove.

11. The instrument according to claim 1, characterized in that the second width (BN) is defined in a manner varying along a length of the recess.

12. The instrument according to claim 1, characterized in that the insulating body is configured in a flexible manner and comprises a first section held inside the groove and a second section projecting out of the groove.

13. The instrument according to claim 12, characterized in that the cutting electrode is arranged in a manner extending through the second section and into the first section.

14. The instrument according to claim 1, characterized in that the recess has a bottom and that the counter support comprises a section that is supported on the bottom of the recess.

15. The instrument according to claim 1, characterized in that the counter support has a section adjoining the sealing electrodes.
